(19) United States
(12) Patent Application Publication
(10) Pub. No.: US 2021/0301138 A1
(43) Pub. Date: Sep. 30, 2021

(54) SYRINGES MADE FROM BIOPLASTIC RESINS

(71) Applicant: Shantu Patel, Carlsbad, CA (US)

(72) Inventor: Shantu Patel, Carlsbad, CA (US)

(21) Appl. No.: 16/834,280

(22) Filed: Mar. 30, 2020

(51) Int. Cl. C08L 99/00; A61M 5/32; A61M 5/31; A61M 5/315; C08L 67/04

(52) U.S. Cl. CPC C08L 99/00; A61M 5/3202; A61M 5/3129; A61M 5/3286; A61M 5/31511; C08L 2207/322; A61M 2205/6072; C08L 2201/06; C08L 2203/02; C08L 2205/03; C08L 2205/06; C08L 67/04

(57) ABSTRACT

A bioplastic resin (BIOSYN) includes corn flour; a biodegradable plasticizer selected from the group consisting of: vegetable oil, polyesters made from glycerin, glycerin, and derivatives of glycerin; and polylactic acid (PLA); further, the bioplastic resin has been subjected to trace element extraction. A second, biodegradable resin includes PLA and acetyl tributyl citrate (ATBC). A syringe includes a bioplastic plunger and needle cap made from BIOSYN, and a biodegradable barrel and tip made from the PLA/ATBC resin.

### BACKGROUND OF THE INVENTION

The present invention generally relates to medical devices made from biodegradable resins and more specifically to syringes made from bioplastic resins.

Environment and sustainability have become increasingly important factors in the design and specification of single use medical items across the world. In hospitals, pharmaceuticals, life sciences, and healthcare industries, safe disposal of articles after use is an important issue. Special considerations are given to selecting materials in the final design for disposable articles, so as to reduce the quantity of syringes items that enter waste streams. These facilities and industries must initiate environmentally safe disposal methods because they generate a large amount of the bio waste. Due to higher social responsibility and environmental concerns, corporations are being driven to produce more sustainable and environmentally safe products through government regulations, by institutional investors, and through consumer demand.

Polylactic acid (PLA) is a transparent bioplastic produced from corn, beet and cane sugar. It not only resembles conventional petrochemical mass plastics, such as polyethylene (PE), polyethylene terephthalate (PET or PETE), and polypropylene (PP) in its characteristics, but it can also be processed easily on standard equipment that already exists for the production of conventional plastics. PLA has a density of 1.25 to 3 g cm, which is lower than PET, and PLA has a refractive index of 1.35-1.45, which is lower than PET, which has a refractive index of 1.54. PLA is currently used in biomedical applications, such as sutures, stents, dialysis media and drug delivery devices. It is also being evaluated as a material for tissue engineering.

Acetyl tributyl citrate (herein referred to as “ATBC”) is a transparent biodegradable plasticizer having low toxicity. It is also referred to as tributyl acetyl citrate, butyl acetylcitratem, O-acetylcitric acid tributyl ester, ATCB, or acetyl tributylcitrate. It has molecular formula C20H34O8. In an embodiment, plasticizers or additives or both may be added to PLA. ATBC can be added to improve flexibility. To overcome distortion, heat tolerant PLA may be used, such as PLLA and PDLA, to offer higher heat distortion properties. If to be used in the human body, all these materials will be of medical grade to meet FDA approval and standards.

Bioplastic resins have some distinct advantages over plastic and glass. Bioplastic has a much smaller carbon footprint compared to plastic or glass, and also uses less energy to form an article like a syringe. Bioplastic is biodegradable in an industrial composting unit. Bioplastic resins are from a plant source, and when plants are grown, they absorb carbon dioxide, thus decreasing carbon dioxide in the atmosphere. Plastic and glass single use items have a higher carbon footprint than bioplastic items.

Currently plastic syringes, when they are disposed of and enter the waste stream, are considered a bio waste. They have to be disposed off in a safe method and may go through incineration. This process is detrimental to the environment in that it causes release of hydrocarbons and toxins into the atmosphere and creates fly ash that ends up in landfills. Bioplastic articles can be steam or chemically sterilized, then shredded and composted, and placed in the fields, thus bypassing the environmentally detrimental process described of plastic syringes. The net impact by using bio based syringes is that it is an environmentally friendly process and leads to near zero waste. Bioplastic articles are environmentally safe and sustainable, when compared to plastic or glass, and leads to near-zero waste. Bioplastic, however, has poor permeability characteristics, in reference to water, oxygen and carbon dioxide. Bioplastic also has poor flexibility properties and is rigid. PLA, a bioplastic, has poor thermal properties, with heat distortion threshold of 55 Celsius, compared to plastics.

It would be desirable to have a syringe that includes a combination of bioplastic and biodegradable resins.

### SUMMARY OF THE INVENTION

In one aspect of the present invention, a bioplastic resin comprises corn flour; a biodegradable plasticizer selected from the group consisting of: vegetable oil, polyesters made from glycerin, glycerin, and derivatives of glycerin; and a biodegradable stiffener; wherein the bioplastic resin has a reduced amount of trace elements, said bioplastic resin having been subjected to trace element extraction.

In another aspect of the present invention, a biodegradable resin comprises polylactic acid (PLA); and acetyl tributyl citrate (ATBC); wherein the biodegradable resin has a reduced amount of trace elements, said biodegradable resin having been subjected to trace element extraction.

In yet another aspect of the present invention, a medical article comprises a bioplastic resin that includes corn flour, a biodegradable plasticizer selected from the group consisting of: vegetable oil, polyesters made from glycerin, glycerin, and derivatives of glycerin, and polylactic acid (PLA), the bioplastic resin having a reduced amount of trace elements, said bioplastic resin having been subjected to trace element extraction; a biodegradable resin that includes PLA, and acetyl tributyl citrate (ATBC), the biodegradable resin having a reduced amount of trace elements, said biodegradable resin having been subjected to trace element extraction.

### BRIEF DESCRIPTION OF THE DRAWINGS

FIG. 1 depicts an embodiment of a syringe with attached needle according to the present invention.

FIG. 2 depicts an embodiment of a needle and safety shield assembly according to the present invention.

FIG. 3 depicts an embodiment of a needle and safety shield in extended position according to the present invention.

FIG. 4 depicts an embodiment of a needle safety shield deployed with needle in the protective casing according to the present invention.

### DETAILED DESCRIPTION

The preferred embodiment and other embodiments, which can be used in industry and include the best mode now known of carrying out the invention, are hereby described in detail with reference to the drawings. Further embodiments, features and advantages will become apparent from the ensuing description, or may be learned without undue experimentation. The figures are not necessarily drawn to scale, except where otherwise indicated. The following description of embodiments, even if phrased in terms of “the invention” or what the embodiment “is,” is not to be taken in a limiting sense, but describes the manner and process of making and using the invention. The coverage of this patent will be described in the claims. The order in which steps are listed in the claims does not necessarily indicate that the steps must be performed in that order.

Embodiments of the present invention may provide disposable syringe with a tubular needle cap and a flexible foldable needle safety shield, and needle hub made from sustainable and environmentally safe bioplastic resins.

Embodiment of the present invention generally provides a bioplastic material comprising bio based materials in the form of corn flour (also called maize or maize flour), and biodegradable plasticizer selected from the group consisting of vegetable oil, polyesters made from glycerin, glycerin, derivatives of glycerin, and combinations thereof.

Embodiments of two medical grade resins, namely PLA/ATBC and BIOSYN, are used to make syringes and related parts including a plunger, barrel, tip and needle protector, and a flexible foldable needle safety shield and needle hub. Embodiments of syringes may be made in different sizes and different lengths. The resins may be used to injection mold or thermoform the parts. In an embodiment, the barrel and tip and flexible foldable needle safety shield and needle hub may be made from PLA ATBC resin and the plunger and needle protector may be made from BIOSYN, by injection mold or thermoforming process.

In an embodiment of a translucent, flexible, biodegradable resin, plasticizers or additives or both may be added to PLA. ATBC can be added to improve flexibility. To overcome distortion, heat tolerant PLA may be used, such as PLLA and PDLA, to offer higher heat distortion properties. If to be used in the human body, all these materials will be of medical grade to meet FDA approval and standards.

In an example, the corn flour comprises a minor volume percent of the total volume of the bio resin. The corn flour may be present in an amount of from about 10 to 45 percent by volume or weight, and biodegradable plasticizer about 22 to 40 percent by volume or weight of the total bio resin. PLA forms the balance 15 to 68 percent by volume or weight.

In an example, the biodegradable plasticizer may be present in an amount of from about 40 to 75 percent by volume or weight, and about 55 to 65 percent by volume or weight of the bio resin. See Table 1.

TABLE 1
Bio resin for syringes

| | Low | High |
|---|---|---|
| Corn | 10% | 45% |
| Bio Plasticizers | 22% | 35% |
| PLA | 68% | 20% |

The bioplastic material may further include a biodegradable stiffener or biodegradable additive such as one useful for adding a desired degree of stiffness to the resulting material, wherein a preferred biodegradable additive is polylactic acid. In an example, the polylactic acid may be present in an amount of from about 15 to 68 percent by volume or by weight of the total bioplastic material. In an example, the bioplastic may include an optional UV stabilizer, e.g., in an amount of from about 6 to 15 percent by volume of the total bioplastic material. These ingredients are mixed and or blended and then polymerized to achieve a resin, called BIOSYN.

An embodiment may include a PLA and ATBC mixture at a ratio of 5 to 35% weight, which produces a bioplastic resin that may be flexible and not rigid, and that may be translucent. The tensile strength of PLA is 36.4 MPa and may be improved to 13.1 MPa, and elongation at break may improve from 7.2% of PLA to 483.5% for the bioplastic mixture. This makes the mixture more flexible.

In an embodiment, PLA or heat stable PLA, namely PLLA, PDLA and additives like ATBC are first subject to trace element extraction, to achieve medical grade resin and additives. To this medical grade PLA is added medical grade ATBC in a ratio from 5 to 35% by weight and mixed in a mixer for 10 minutes and heat dried. Similarly, BIOSYN resin is subject to trace element extraction derived for corn flour, and biodegradable plasticizer selected from the group consisting of vegetable oil, polyesters made from glycerin, glycerin, derivatives of glycerin, and PLA to achieve a medical grade product. This final medical grade resins and granules are then bagged and a batch and a traceable code are printed on each bag and detailed log is kept.

This embodiment of a bioplastic consists of a syringe with attached needle and foldable and extendable protective safety shield assembly. Embodiments of bioplastic syringes may be used for insulin administration, allergy or tuberculin testing or administration of other parental agents. The syringe may have a plunger that fits tightly in a tubular barrel with a tip. The plunger may be pulled and pushed along inside a cylindrical tube, the barrel, allowing the syringe to take in and expel a liquid or gas through an orifice at the open end of the tube called the tip. The open end of the syringe or the tip may be fitted with a hypodermic needle. The tip can of various shapes, but usually cone shape or threaded to allow for a needle or tubing to be attached, by simple pushing or screwing, the latter by circular twisting motion. This attachment will help direct the flow into and out of the barrel. The needle head is called a hub and an opening or aperture. Embodiments of bioplastic syringes may be used in the medical field to administer injections, insulin administration, skin tests such as allergy tests, and tuberculin testing. In non-medical field uses, non-sterile bioplastic syringes may be used to apply compounds such as glue or lubricant, and measure liquids.

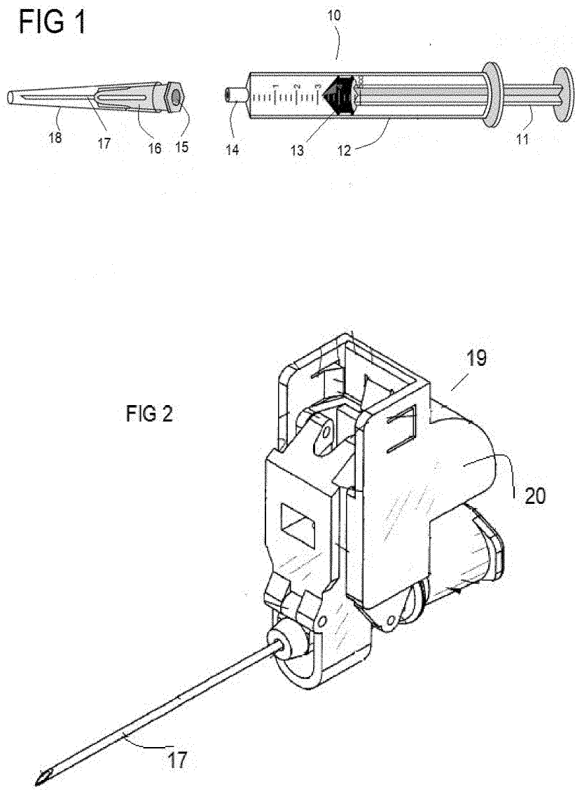

FIG. 1 depicts a perspective view of a general propose disposable single-use syringe 10 that may has a plunger 11 with a rubber tip 13, a cylindrical barrel 12, a hypodermic needle 17 and a needle cap 18. Plunger 11 and needle cap 18 may be made from Biosyn resin. The barrel 12 and attached tip 14 may be made from PLA/ATBC resin. The needle 17 may have a hub 16 and opening or aperture 15. The hub 16 may be made from PLA or Biosyn resin.

FIG. 2 depicts needle and safety shield assembly 19, which may include a needle 17 and a shield assembly 20 made from PLA/ATBC.

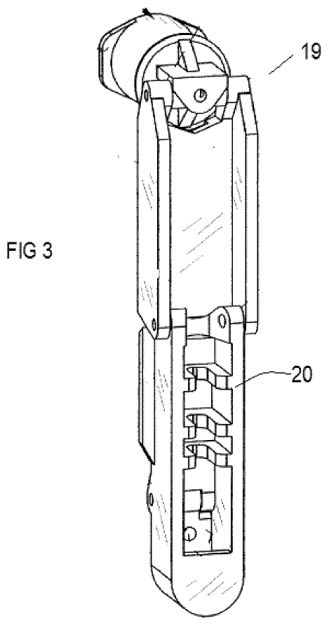

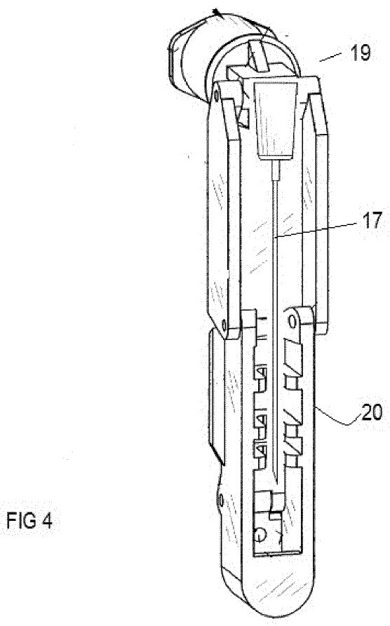

FIG. 3 depicts an embodiment of a safety needle and shield assembly 19 without the needle in an extended position.

FIG. 4 depicts an embodiment of a safety needle and shield assembly 19 with the needle in an extended position, which may accommodate the needle in a protective casing.

An embodiment of a syringe safety needle assembly 19 may include a syringe 10, fixed to a medical or hypodermic needle 17. An embodiment of a bioplastic cap and a safety shield assembly 20 may attach to the syringe needle assembly 19, to help provide protection from a sharpened tip of the needle 17. The cap 18 may be removed before using the syringe needle assembly. After using the syringe needle assembly 19, the safety shield 20 may be deployed. The safety shield 19 may be foldable or may include a tubular assembly 18, thereby providing a safety sheath for the needle. This may help guard against problems associated with inadvertent needle sticks related to blood sampling, percutaneous medication injection and other medical procedures involving uses of medical needles.

I claim:

1. A bioplastic resin comprising:
corn flour;
a biodegradable plasticizer selected from the group consisting of: vegetable oil, polyesters made from glycerin, glycerin, and derivatives of glycerin; and
a biodegradable stiffener;
wherein the bioplastic resin has a reduced amount of trace elements, said bioplastic resin having been subjected to trace element extraction.

2. The bioplastic resin of claim 1, wherein the biodegradable stiffener is polylactic acid (PLA).

3. The bioplastic resin of claim 2, wherein
the corn flour is from 10% to 45% by weight of the total,
the biodegradable plasticizer is from 22% to 35% by weight of the total, and
the PLA is the remainder of the weight of the total.

4. The bioplastic resin of claim 1, wherein
the bioplastic resin s formed into a bioplastic plunger for a syringe.

5. The bioplastic resin of claim 1, wherein
the bioplastic resin is formed into a bioplastic needle cap for a syringe.

6. The bioplastic resin of claim 1, wherein
the bioplastic resin is formed into a bioplastic needle hub for a syringe.

7. A biodegradable resin comprising:
polylactic acid (PLA); and
acetyl tributyl citrate (ATBC);
wherein the biodegradable resin has a reduced amount of trace elements, said biodegradable resin having been subjected to trace element extraction.

8. The bioplastic resin of claim 7, wherein
the ratio of PLA to ATBC mixture is 5% to 35% by weight.

9. The biodegradable resin of claim 7, wherein
the biodegradable resin is formed into a biodegradable barrel for a syringe.

10. The biodegradable resin of claim 7, wherein
the biodegradable resin is formed into a biodegradable tip for a syringe.

11. The biodegradable resin of claim 7, wherein
the biodegradable resin is formed into a biodegradable foldable needle safety shield for a syringe.

12. A medical article comprising:
a bioplastic resin that includes
corn flour,
a biodegradable plasticizer selected from the group consisting of: vegetable oil, polyesters made from glycerin, glycerin, and derivatives of glycerin, and
polylactic acid (PLA),
the bioplastic resin having a reduced amount of trace elements, said bioplastic resin having been subjected to trace element extraction; and
a biodegradable resin that includes
PLA, and
acetyl tributyl citrate (ATBC),
the biodegradable resin having a reduced amount of trace elements, said biodegradable resin having been subjected to trace element extraction.

13. The medical article of claim 12 further comprising:
a bioplastic syringe plunger that includes the bioplastic resin; and
a biodegradable syringe barrel that includes the biodegradable resin.

14. The medical article of claim 12, wherein the medical article is a syringe that further comprises:
a bioplastic syringe plunger that includes the bioplastic resin;
a bioplastic syringe needle cap that includes the bioplastic resin;
a biodegradable syringe barrel that includes the biodegradable resin; and
a biodegradable syringe tip that includes the biodegradable resin.

15. The medical article of claim 12, wherein the medical article further comprises a bag and a traceable code printed on the bag that identifies a batch of the bioplastic resin and a batch of the biodegradable resin.
